(19) United States
(12) Patent Application Publication
(10) Pub. No.: US 2025/0179286 A1
(43) Pub. Date: Jun. 5, 2025

(54) POLYZWITTERIONIC COMPLEXES AND METHODS OF USING THE SAME

(71) Applicants: Khatcher Orbeli Margossian, Amherst, MA (US); Marcel Brown, Amherst, MA (US); Todd Emrick, Amherst, MA (US); Murugappan Muthukumar, Amherst, MA (US)

(72) Inventors: Khatcher Orbeli Margossian, Amherst, MA (US); Marcel Brown, Amherst, MA (US); Todd Emrick, Amherst, MA (US); Murugappan Muthukumar, Amherst, MA (US)

(21) Appl. No.: 18/845,131

(22) PCT Filed: Mar. 9, 2023
(86) PCT No.: PCT/US2023/064002

§ 371 Date: Sep. 9, 2024

Related U.S. Application Data
(60) Provisional application No. 63/318,760, filed on Mar. 10, 2022.

(51) Int. Cl. C08L 43/02; A61K 9/50

(52) U.S. Cl. CPC C08L 43/02; A61K 9/5031

(57) ABSTRACT

The present teachings relate to a polymer system including a coacervate or precipitate formed through dipole-charge interactions or dipole-dipole interactions such as between a polyelectrolyte and a polyzwitterion, which form a polyzwitterionic complex. The polymer system can be used to deliver an encapsulated cargo, for example, a therapeutic drug product, which is released when the ambient pH increases.

### CROSS-REFERENCE TO RELATED APPLICATION

This patent application claims the benefit of and priority to U.S. Patent Application No. 63/318,760 filed on Mar. 10, 2022, the entire contents of which are incorporated by reference herein.

### FIELD

A coacervate or precipitate formed through dipole-charge interactions or dipole-dipole interactions, for example, a polyzwitterionic complex that is formed by a polyelectrolyte and a polyzwitterion. Such complexes can encapsulate and deliver a cargo based on changes in the ambient environment, for example, pH.

### BACKGROUND

Complex coacervates can be formed by the association of two oppositely-charged polyelectrolytes. Their self-assembly can be driven by the entropically favourable release of counterions and solvent into the bulk mixture. This process can result in liquid-liquid phase separation between a polymer-rich dense phase, and a polymer-poor dilute supernatant. When viewed using optical microscopy, the hallmark of coacervation can include the presence of smooth, mobile, and coalescing spherical droplets. Complex coacervation can include biologically-inspired monomers that form specific three-dimensional structures, incorporation of charged groups into block copolymers, sequential and chiral patterning, and many other physicochemical alterations. Complex coacervation can include the association of two macromolecular chains that are of uniformly negative or positive charge.

Classically, coacervate solutions can be stable within a pH range that allows both cationic and anionic species to remain in a maximally charged state. This phenomenon can be illustrated by the top box in FIG. 1(a), which denotes the range in which coacervates are stable in some polyelectrolyte systems. Outside this range, there can be insufficient ionization of one chain to promote coacervation. However, it can be desirable to push this box to different ranges, for example in order to control the retention and release characteristics using pH as a chemical trigger. Moving the box leftwards (FIG. 1(a), bottom box) to lower pH ranges can present opportunities in the physiological arena.

An example in which such a scenario is beneficial is in the gastrointestinal (“GI”) tract. Considering the large pH gradient between the stomach and the intestines (FIG. 1(b)), platform technologies that control the retention and release of material using this large gradient can improve the delivery of cargo to areas distal to the stomach. This problem can be of great medical interest because currently, many oral drug delivery technologies suffer from significant degradation upon exposure to the harsh gastric environment. As a result, many modern therapeutics cannot be formulated as orally delivered drugs and can instead must be delivered intravenously. Avoiding gastric degradation can be tantalizing because the small intestine is the site of greatest and most efficient absorption in the GI tract. Furthermore, intestinal conditions can be much gentler, since the production of basic secretory fluids can neutralize its luminal pH.

Thus, there is a need in the art for drug delivery technologies to take advantage of this significant pH differential, for example, by using it as a chemical trigger.

There is also a need in the art for acid-resistant coatings for containers storing and/or transporting acidic liquids.

### SUMMARY

Complex coacervation can be regarded as a process whereby two oppositely charged polyelectrolytes self-assemble into spherical droplets. In contrast, the present teachings relate to systems and methods that include a polyzwitterionic complex (or “pZC”) which can be formed by the liquid-liquid phase separation of a polyzwitterion and a polyelectrolyte. This system can exhibit orthogonal phase behaviour. For example, it can remain intact in acidic conditions, but disassemble as the pH increases. This process can be governed by the acid-base equilibria of the constituent chains. The observed phase behaviour can be related to physiological conditions within the GI tract with a simulation of the gastroduodenal junction. The viability of polyzwitterionic coacervates as technologies for the pH-triggered release of cargo can be demonstrated using video microscopy. Such a system can tackle imminent problems of drug transport via the oral route and can serve as a packaging solution to increase uptake efficiency.

Features of pZCs lend themselves to drug delivery vehicles that can deliver their cargo based on changes in ambient pH, for example, a drug delivery vehicle that can remain intact under low pH conditions such as in the stomach but delivers its cargo when at a higher pH, for example, in the small intestine.

In one aspect, the present teachings provide a polymer system comprising a coacervate or precipitate formed through dipole-charge interactions or dipole-dipole interactions. The dipole-charge interactions or dipole-dipole interactions can occur between a polyelectrolyte and a polyzwitterion. For example, the polymer system can include a polyelectrolyte and a polyzwitterion, whereby the polyelectrolyte and the polyzwitterion form a polyzwitterionic complex.

In some embodiments, the polyzwitterionic complex encapsulates a cargo, such as a therapeutic drug product. The therapeutic drug product can be selected from the group consisting of insulin, a probiotic agent, a non-steroidal anti-inflammatory drug, a vaccine, a monoclonal antibody, and a nucleic acid. In certain embodiments, the polyelectrolyte is a pharmaceutically acceptable polyelectrolyte and the polyzwitterion is a pharmaceutically acceptable polyzwitterion.

In various embodiments, the polyzwitterion is grafted to a substrate. In such embodiments, the polyzwitterionic complex, after formed, can cover the substrate and protect it, for example, from acidic conditions and/or decomposition.

The polyelectrolyte can be selected from the group consisting of poly(acrylic acid), poly(styrene sulfonic acid), poly(methyl methacrylate), poly(dimethyl amino ethyl methacrylate), poly(glutamic acid), poly(aspartic acid), heparan sulfate, hyaluronic acid, and co-polymers thereof. The polyzwitterion can be selected from the group of polyzwitterion classes consisting of a phosphorylcholine class, a sulfobetaine class, a carboxybetaine class, an ammonium sulfonate class, a sulfopyridinium betaine class, a cysteine class, and co-polymers thereof. In particular embodiments, the polyzwitterion is poly(2-methacryloyloxyethyl phosphorylcholine). In some embodiments, at least one of the polyelectrolyte and the polyzwitterion is a co-polymer.

In various embodiments, the polymer system further comprises a solvent. The solvent can be water, methanol, ethanol, ethylene glycol, propylene glycol, polyethylene glycol, or combinations thereof. Further, the solvent can include one or more of sodium chloride, potassium chloride, a sugar, hydrochloric acid, sulfuric acid, nitric acid, hydrobromic acid, and acetic acid.

In some embodiments, the polymer system is in a solid state. The polymer system in the solid state can include a cargo. In certain embodiments, the present teachings provide a reconstituted polymer system comprising a polymer system as described herein; and water.

A polymer system or a reconstituted polymer system as described herein can form the polyzwitterionic complex at a pH of about 1 to about 3.

In another aspect, the present teachings provide a method of using a polymer system to deliver a cargo, where the polymer system comprises a polyzwitterionic complex formed from a polyelectrolyte and a polyzwitterion. The polymer system can encapsulate a cargo whereby when the pH of the surrounding environment changes, the polyzwitterionic complex is undone thereby releasing the encapsulated cargo. In various embodiments, the pH of the surrounding environment is increased to release the encapsulated cargo such as a therapeutic drug product.

In some embodiments, a method of creating a layer or coating of polyzwitterionic complexes is provided whereby the coated substrate includes an acid-resistant layer. In certain embodiments, a polyzwitterion is tethered or grafted to a substrate, which is then dipped into a polyelectrolyte solution at a low pH to create the polyzwitterionic complexes. Such a coating could be useful for transporting acidic liquids in coated containers, or for protecting boats hulls from seawater and its contaminants.

### DESCRIPTION OF DRAWINGS

It should be understood that the drawings described below are for illustration purposes only. The drawings are not necessarily to scale, with emphasis generally being placed upon illustrating the principles of the present teachings. The drawings are not intended to limit the scope of the present teachings in any way.

FIGS. 1(a) and 1(b): Schematics of the orthogonal phase behaviour of polyzwitterionic complexes and their potential applicability to GI cargo delivery. (a) Comparing the phase behaviour of some polyelectrolyte complexes (top box) with polyzwitterionic complexes (bottom box). In some systems, phase stability (i.e., the parameter space in which stable complexation can occur) is present at intermediate pH values (top box) because of the pKₐ values of the constituent chains. Maximal complexation can occur when both positive and negative chains are maximally charged, typical values for which can include those found within the top box. In contrast, with polyzwitterionic complexes, stable spherical droplets can form when the pH is in a more acidic range (bottom box). (b) Schematic diagram of pH variation along the GI tract and design principle. Inside the acidic environment of the stomach, polyzwitterionic complexes can remain in their associated state. As they move into the duodenal space, the pH trace can rapidly increase to a value of 6-8, which can result in de-complexation.

FIGS. 2(a)-2(f): (a) At low pH (˜2), the repeat unit of poly(acrylic acid) (or “pAA”) can be deionized and can act as a dipole, while the phosphoryl group of the polyzwitterionic repeat unit of poly(2-methacryloyloxyethyl phosphorylcholine) (or “pMPC”) can be deionized so that the monomer can act as a positive charge, resulting in dipole-charge interaction at separation distance r_{dc}. This attractive energy, in combination with counterion release, can lead to complexation. (b) Schematic of the electrostatic interaction between pAA and pMPC at pH˜4. The pAA monomer can be deionized and can act as a dipole. However, the phosphoryl group of pMPC can be ionized so that the monomer is electrically neutral and thus, also can act as a dipole. The net effect can be a weak dipole-dipole interaction between pAA and pMPC. (c) Since the pKₐ of the phosphoryl group is 2.3 and the charge on the ammonium group is fixed, the net charge Qₚz of the polyzwitterion can change from +1 to zero as the pH is increased from 2 to 4. (d) Snapshot of the packing model of a repeat unit of pAA and that of pMPC from the Avogadro software. The closest distance between the dipolar pAA and the charged pMPC is 0.38 nm; for the dipolar polyzwitterion it is 0.59 nm. (e) Plot of the dipole-charge interaction energy per contact (curved trace) as a function of their separation distance (I_{B}=0.7 nm, p₁=0.0354, and κ=0.329 nm⁻¹ at pH 2). The vertical line represents the nearest distance between the charge and the dipole, 0.38 nm. (f) Plot of dipole-dipole interaction energy per contact (curved trace) as a function of their separation distance; 1_{B}=0.7 nm, p₁=0.0354, p₂=0.39, κ=0.0329 nm⁻¹ at pH 4. The vertical line represents the nearest distance between dipoles, 0.59 nm.

FIGS. 3(a) and 3(b): Materials used in the example: (a) 22 kDa pMPC was synthesized from monomeric MPC, and mixed with (b) pAA of 50 kDa molecular weight to form complexes.

FIGS. 4(a) and 4(b): Stoichiometry of pMPC:pAA vs. turbidity at pH 2 (squares) and 3 (circles), plotted over a temperature range from 20° C.-40° C. (a) Data from the pH 2 samples demonstrate a peak value at a 30:70 mixing ratio. By comparison, the pH 3 data at the bottom of graph (a) exhibit a much smaller optical density across the range of stoichiometries. (b) Magnification of data at pH 3 as shown in FIG. 4(a), with a peak value at a 60:40 mixing ratio. This optical density peak is much lower, but is present across a broader range of stoichiometric ratios. Error bars represent the standard deviation between measurements

FIGS. 5(a) and 5(b): Microscopic appearance of pZCs at pH 2 and 3. ((a), left) Complexes with spherical morphology at pH=2. Images can be taken with a polarized objective lens at 10× magnification. This is a representative image, corresponding to the letter A in FIG. 6, a 30:70 pMPC:pAA stoichiometric ratio. Scale bar corresponds to a length of 120 μm. There can be a resemblance to some polyelectrolyte complexes. ((b), right) Complexes with spherical morphology at pH=3. Images can be taken with the same polarizer objective lens at 10× magnification. This is a representative image, corresponding to the letter B in FIG. 6, a 60:40 pMPC:pAA stoichiometric ratio. Scale bar corresponds to a length of 100 μm. Complexation in these conditions can be much less abundant than in those seen in (a).

FIG. 6: Compiled phase diagram comparing mixing stoichiometry (% pMPC of the total mixture, abscissa) with solution pH (ordinate), taken from experiments like those of FIGS. 4(a) and 4(b) and 5(a) and 5(b). Colour axis indicates the value of the turbidity. Minimal to no complexation can occur above and outside the black dotted line as determined by optical microscopy. Regions in darker grey near the letter “A” represent the greatest levels of complexation, and those regions outside the dotted line represent no detectable complexation. Results depicted in this diagram can be consistent with findings from microscopy. An optical micrograph image of the sample marked by the letter A is seen in FIG. 5(a), and the sample corresponding to the letter “B” is seen in FIG. 5(b).

FIGS. 7(a) and 7(b): Mechanism for pH-dependent phase behavior of pMPC-pAA complexes. Dipolar pAA chains are depicted with arrows, and the polyampholyte pMPCs are drawn with positive charges along its chain. The corresponding charges of each chain are drawn as negative symbols and positive symbols. In the case of pMPC, charges can be tethered to monomers further from the chain backbones, and are therefore drawn as appendages, with the negative charge proximal to the backbone, and the positive charge distal to it. pAA chains can be more closely approximated simply as a line of dipoles due to their smaller monomer size, and much larger chain length. ((a), left) pH less than 4: The ionization equilibrium can favour the protonation of the pMPC phosphate groups, which confers a formal charge of +1 for each pMPC monomer. In this scenario, the exposed positive charges on pMPC can interact with dipoles along the pAA chains, promoting complexation, and accompanied by counterion release. ((b), right) pH greater than 4: the phosphoryl groups of the pMPC can be mostly deprotonated, effectively neutralizing the positive charge on the choline group. In this state, the net charge of each pMPC monomer can be zero, and thus there may not be favourable interaction promoting association with pAA, owing to the relative weakness of the dipole-dipole interaction compared to the charge-dipole interaction.

FIGS. 8(a)-8(c): Effects of pH and temperature on pZC assembly. (a) Scattered light intensity as a function of solution pH of 50:50 stoichiometric ratio pMPC:pAA. (b) At pH 2, in samples with a 30:70 stoichiometric ratio (corresponding to the peak), heating over a temperature ranging from 20° C.-40° C. can cause negligible changes in coacervate turbidity, which can demonstrate stability of pMPC:pAA complexes with respect to temperature. (c) Peak samples at pH 3 (60:40 pMPC:pAA stoichiometric ratio) can exhibit a similar resistance against thermal fluctuations over the same 20° C.-40° C. range. Error bars represent the standard deviation between measurements.

FIG. 9: Physical simulation to expose pZCs to physiological conditions relevant to the duodenum. Addition of water to the edge of the droplet (top, circular dot) can allow wetting of glass surface along a line near the circumference of the pZC mixture (depicted as a line partially under the pZC mixture in the second picture down). Next, a small volume of NaOH solution (circular dot further from pZC mixture in third picture down) can be micropipetted at the edge of the wet line created previously. This system in turn can promote the orderly movement of NaOH into the polymer mixture unidirectionally and gradually.

FIGS. 10(a)-10(h): Compilation of video microscopy image frames of NaOH dissolving the pMPC-pAA complexes. A very small volume of NaOH solution can be dropped beside a drop of the polymer mixture (in sandy-looking area), and the resulting solvent front (also in sandy-looking area) can be recorded using a polarized objective with 10× magnification. Frames at 2 second intervals can be captured using the Phantom PCC imaging software, and presented in order from FIG. (a) to FIG. (h). The scale bar in FIG. (a) can correspond to a length of 125 μm, and can be consistent throughout the frames.

FIG. 11: Absorbance spectrum of AlexaFluor-tagged BSA. An absorbance peak at approximately 495 nm can indicate the presence of the fluorescently-labeled protein. Note the absence of absorbance at 550 nm, can allow the presence of protein and polymer to be tracked independently using absorbance spectroscopy.

FIG. 12: Bulk solutions of BSA-loaded pZCs can have the highest absorbance at low pH, consistent with previous results. As pH increases, the pZC assemblies can dissolve, and thus their absorbance can fall to baseline levels. The presence of pZCs can be monitored by the absorbance at 550 nm (diamonds). Absorbance values at 495 nm (circles) can be used to track the tagged BSA. As the pH increases and the complexes dissolve, the protein absorbance can decrease to its baseline level of approximately 0.2 a.u. This value can be much higher than the baseline reading for the polymers at 550 nm, which can be between 0.04 and 0.05 a.u.

FIG. 13: After centrifugation of the BSA-loaded pZCs, the supernatant absorbance can be measured as a function of pH. The absorbance at 550 nm (squares) can be close to baseline regardless of the measurement, indicating that the polymer assemblies may not be present in the supernatant. As the pH increases, the pZCs can disassemble. They may not form complexes at pH 4 or 5, but at pH 2 and 3, the complexes they do form can be effectively excluded from the measurement, as indicated by the relatively flat values of the 550 nm absorbance measurements. However, as the pZCs disassemble, the absorbance values at 495 nm (triangles) can increase steadily, until they reach the 0.2 a.u. value that may be expected when they are free in solution. (This is the peak absorbance value seen in the initial absorbance curve.) The sub-0.2 a.u. value before pH 5 can indicate that there may be some degree of interaction between the polymer chains and the protein in acidic conditions. Nevertheless, the increasing absorbance as a function of increasing pH can indicate that BSA is preferentially segregating with the polymer complexes at low pH, and can be released as complexes dissolve at higher pH values. The complete absence of BSA absorbance at pH 2 can show that this cargo can interact closely with its carrier, and that as the carrier is made to dissolve, the protein cargo can be released into the bulk.

FIG. 14: Fluorescence micrograph of AlexaFluor488-conjugated BSA encapsulated within pZC droplets at pH 2. Image can demonstrate preferential segregation of the protein into the polymer-rich phase, and absence of the protein in the polymer-poor phase.

FIG. 15: The cross-polarization (“CP”) spectrum with magic angle spinning speed of 7 kHz and CP time of 1 ms. The 55 ppm peak and the 17 ppm peak are from N-methyl and the backbone methyl in MPC, respectively. The 180 ppm peak can be from the carbonyl signal (present in both PAA and MPC), while the small peaks at 130 and 225 ppm can be spinning sidebands of the carbonyl. Other peaks can also be appropriately assigned. The backbone methyl instead of the N methyl can be used to represent the MPC population as the former can have similar molecular dynamics as the PAA carbonyl and thus would be excited to a similar degree in CP experiments. The presence of these peaks can indicate that the complexes contain both the pAA and pMPC components.

FIG. 16: NMR spectrum and associated shifts of pMPC. ¹H-NMR (500 MHz, D₂O, δ): 8.05-7.50 (CTA), 4.40-4.00 ppm (6H), 3.71 ppm (2H), 3.27 ppm (9H), 2.20-0.80 (5H+CTA).

### DETAILED DESCRIPTION

The systems and methods of the present disclosure can be based on the theory of complexation between charged macromolecules. Two major contributions to inter-molecular complexation among polar and charged macromolecules can include electrostatic interactions among all polar and charged repeat units of the complexing chains and entropy gain due to the release of counterions during complexation. Complexation between a host macromolecule and a guest macromolecule can be considered. If both host and guest are uniformly charged as in the case of complexation between polycations and polyanions, the electrostatic interaction can include the strongly attractive charge-charge interaction, with counterion release further augmenting the process. On the other hand, if the host is made of essentially polar repeat units with only very few charges, then it can be treated as a chain of dipoles. If the guest is uniformly charged, the electrostatic interaction between the dipolar host and charged guest can include a dipole-charge interaction. Complexation in this situation too can be facilitated by the dominant driving force from counterion release. If both the host and guest are dipolar chains, then the electrostatic interaction energy can include a dipole-dipole interaction, which can be much weaker than the dipole-charge interaction. Furthermore, there can be no additional driving force from counterion release if both the host and guest are dipolar. Therefore, the spontaneously formed coacervate complex from a dipolar chain and a charged chain can dissolve when the condition is such that the dipole-charge interaction is switched to a dipole-dipole interaction.

Accordingly, the present teachings describe a coacervate or precipitate formed through dipole-charge interactions and/or dipole-dipole interactions. The coacervate or precipitate can include a polyelectrolyte and a polyzwitterion. For example, the polyelectrolyte and the polyzwitterion can form a polyzwitterionic complex (or pZC).

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

The terms “a” and “an” as used herein mean “one or more” and include the plural unless the context is inappropriate.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10%, ±5%, ±3%, or ±1% variation from the nominal value unless otherwise indicated or inferred from the context.

At various places in the present specification, values are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As used herein, “pharmaceutically acceptable” refers to compounds, molecular entities, compositions, materials, and/or dosage forms that do not produce an adverse, allergic or other untoward reaction when administered to an animal, or a human, as appropriate. For human administration, preparations should meet sterility, pyrogenicity, general safety and purity standards as required by FDA Office of Biologics standards.

As used herein, “pharmaceutically acceptable excipient” refers to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxypropylmethylcellulose, polyvinyl pyrrolidine, and colors, and the like. Such preparations can be sterilized and, if desired, mixed with auxiliary agents such as lubricants, preservatives, stabilizers, surfactants (e.g., polysorbate 20 or polysorbate 80), wetting agents, emulsifiers, salts for influencing osmotic pressure, buffers, coloring, and/or aromatic substances and the like that do not deleteriously react with the compounds of the invention. For examples of excipients and carriers, see Martin, Remington's Pharmaceutical Sciences, 15th Ed., Mack Publ. Co., Easton, PA (1975).

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### Polyzwitterion Complexes

The present teachings provide in its broadest sense a polymer system comprising a coacervate or precipitate formed through dipole-charge interactions or dipole-dipole interactions. The dipole-charge interactions or dipole-dipole interactions can occur between a polyelectrolyte and a polyzwitterion. For example, the polymer system can include a polyelectrolyte and a polyzwitterion, whereby the polyelectrolyte and the polyzwitterion form a polyzwitterionic complex.

A polyzwitterionic complex is typically formed at low pH between a polyelectrolyte and a polyzwitterion. The polyelectrolyte is usually neutrally charged across the relevant pH regime. Alternatively, the polyzwitterion has a positive charge at low pH, but when the pH increases it becomes more negatively charges thereby causing disassociation of the complex formed between the polyelectrolyte and polyzwitterion. If the complex is encapsulating a cargo such as a therapeutic drug product, it can be delivered orally so that the complex disassociates at the higher pH of the GI tract, avoiding the need to deliver the therapeutic drug product via injection to by-pass the stomach. Indeed the polyelectrolyte and polyzwitterion at low pH form droplets that encapsulate the cargo for delivery.

In some embodiments, the therapeutic drug product can be selected from the group consisting of a diabetes medication, insulin, a probiotic agent, a non-steroidal anti-inflammatory drug, a vaccine, a monoclonal antibody, a nucleic acid, and a combination thereof. A diabetes medication can include insulin, a glucagon-like peptide-1 (GLP-1) receptor agonist, or a gastric inhibitory peptide (GIP). The therapeutic drug product usually is one in which significant degradation occurs upon exposure to the harsh gastric environment. To avoid intravenous delivery, the therapeutic drug product can be formulated as an orally-deliverable drug product using the pZCs of the present teachings. That is, delivery of the therapeutic drug product in the small intestine, bypassing the stomach, is also beneficial because the small intestine is the site of greatest and most efficient absorption in the GI tract. Accordingly, it should be understood that in certain embodiments, the polyelectrolyte is a pharmaceutically acceptable polyelectrolyte and the polyzwitterion is a pharmaceutically acceptable polyzwitterion.

In some embodiments, the polymer system is formed into or is in the form of a solid state, for example, by lyophilization of the pZC-rich layer after complex formation encapsulating the cargo. The polymer system in the solid state can include a cargo such that a therapeutic drug product can be stored in the solid state for direct delivery into the stomach with water. In certain embodiments, the solid state of the polymer system is reconstituted with water and optionally including a pharmaceutically acceptable acid or other pharmaceutically acceptable excipient, as needed. In such cases, the reconstituted polymer system as described herein can have a pH of about 1 to about 3.5 or of about 2 to about 3, thereby forming the polyzwitterionic complex.

In some embodiments, in the process of making the pZCs of the present teachings, the pH of the solution can be from about 0.5 to about 5, from about 1 to about 4, from about 1 to about 3 from about 2 to about 3, or from about 2.5 to about 3.5.

When not in the solid state or when forming a pZC including a therapeutic drug product, the polymer system further includes a solvent. The solvent can be water, methanol, ethanol, ethylene glycol, propylene glycol, polyethylene glycol, or combinations thereof. Further, the solvent can include one or more of sodium chloride (NaCl), potassium chloride (KCl), a sugar, hydrochloric acid (HCl), sulfuric acid, nitric acid, hydrobromic acid, and acetic acid (AcOH). For therapeutic drug product delivery, pharmaceutically acceptable excipients such as NaCl, KCl, a sugar, HCl, and AcOH can be used.

The polyelectrolyte can be selected from the group consisting of poly(acrylic acid) or pAA, poly(styrene sulfonic acid), poly(methyl methacrylate), poly(dimethyl amino ethyl methacrylate), poly(glutamic acid), poly(aspartic acid), heparan sulfate, hyaluronic acid, and co-polymers thereof. In some embodiments, the polyelectrolyte is pAA.

The polyzwitterion can be selected from the group of polyzwitterion classes consisting of a phosphorylcholine class, a sulfobetaine class, a carboxybetaine class, an ammonium sulfonate class, a sulfopyridinium betaine class, a cysteine class, and co-polymers thereof. In particular embodiments, the polyzwitterion is poly(2-methacryloyloxyethyl phosphorylcholine) or pMPC.

In some embodiments, at least one of the polyelectrolyte and the polyzwitterion is a co-polymer.

In various embodiments, the polyzwitterion is grafted to a substrate. In such embodiments, the substrate then can be contacted with a polyelectrolyte solution, for example, dipped into a polyelectrolyte solution at low pH (e.g., 0.5-5) to form the polyzwitterionic complex and provide a robust acid-resistant layer over the substrate. These layers could be used to coat the interior of containers for transporting acidic liquids. Alternatively, such layers could be useful on the hulls of water vessels to protect from seawater and associated contaminants.

For example, consider a polymer system designed for oral delivery of an acid-sensitive therapeutic drug product, where the host molecule is poly(acrylic acid) (pKₐ′ 6.2) and the guest molecule is the polyzwitterion pMPC (pKₐ′ 2.3 for the phosphoryl group). Under the experimental conditions of interest for therapeutic drug delivery through the stomach (e.g. pH less than 4), polyacrylic acid can be treated as a dipolar chain. For pH less than 4, the polyzwitterion can essentially be a polycation, and for pH greater than 4, it can be a dipolar chain. Since the dipole-charge interaction, accompanied by counterion release, can result in complexation and mere dipole-dipole interactions may not be strong enough to lead to complexation, a mechanism of devising a change from a complexed state to an uncomplexed state in the above mentioned pH range can be envisaged, by making the polyzwitterionic repeat unit of the guest charged at lower pH and dipolar at higher pH.

Quantitative computation of the energetics associated with coacervate complexation involving polyzwitterions can be difficult due to rich chemical details and the lack of knowledge about the local dielectric constant that mediates the various electrostatic interactions in the system. However, a qualitative and instructive account of the energetics for the above specific system can be formulated as follows. Focusing on the neighbourhood of a pair of complementary repeat units, the situations corresponding to low pH and high pH can be schematically shown in FIGS. 2(a) and 2(b), respectively. At both low pH and high pH considered here, the acrylic acid repeat unit can be treated as a dipole with dipole moment \vec{P}₁. However, at low pH, the polyzwitterionic repeat unit can carry a net positive charge due to protonation of the phosphoryl group; at higher pH, the polyzwitterionic repeat unit can be a dipole with dipole moment \vec{P}₂. The net charge of the MPC group (Q_{pz}) can be sketched in FIG. 2(c) as a function of pH. Therefore, the electrostatic energy of complexation at low pH can include the dipole-charge interaction energy (v_{dc}) and that at higher pH can include the dipole-dipole interaction energy (v_{dd}). Averaging over all allowed orientations of the dipoles separated by distance r, v_{dc}(r) and v_{dd}(r) can be given by

$$\frac{{v}_{d c} ( r )}{{k}_{B} T} = - \frac{1}{6} \frac{{ℓ}_{B}^{2} {p}_{1}^{2}}{{r}^{4}} {{e}^{- 2 κ r} ( 1 + κ r )}^{2} , \qquad ( 1 )$$

$$and$$

$$\frac{{v}_{dd} ( r )}{{k}_{B} T} = - \frac{1}{3} \frac{{ℓ}_{B}^{2} {p}_{1}^{2} {p}_{2}^{2}}{{r}^{6}} {e}^{- 2 κ r} [ 1 + 2 κ r + \frac{5}{3} {( κ r )}^{2} + \frac{2}{3} {( κ r )}^{3} + \frac{1}{6} {( κ r )}^{4} ] , \qquad ( 2 )$$

where k_{B}T is the Boltzmann constant times the absolute temperature, l_{B} is the Bjerrum length (=e²/(4πϵ₀k_{B}T), e is the electronic charge, E₀ is the permittivity of vacuum, is the effective dielectric constant of the medium), and κ is the inverse Debye length.

A model of the nearest distances between the acrylic acid dipole and charged ammonium group and that between the acrylic acid group and the dipolar polyzwitterion can be given in FIG. 2(d), based on the Avogadro software. Interaction energies can be minimized from numerous starting configurations using the MMFF-94 (Merck Molecular Force Field) algorithm, and the calculated geometrical configurations can approximate global minima. As an estimate, these distances can be taken to be 0.38 nm and 0.59 nm, respectively. Taking 1_{B}=0.75 nm (corresponding to aqueous solutions at room temperature), κ=0.329 nm⁻¹ at pH 2 and κ=0.0329 nm⁻¹ at pH 4, p₁/e=0.0354 nm (corresponding to the dipole moment of acrylic acid as 1.7 D), and estimating the dipole moment of dipolar polyzwitterion as 0.39 e (corresponding to 18.74 D), typical r-dependence of v_{dc} and v_{dd} calculated from Equations (1) and (2) are shown in FIGS. 2(e) and 2(f). Here the cutoff distances for v_{dc} and v_{dd} can be taken from FIG. 2(d) as 0.38 nm and 0.59 nm, respectively.

As seen from FIGS. 2(e) and 2(f), the pairwise interaction energy of a charge and a dipole can be an order of magnitude stronger than that for a pair of dipoles. The latter can be much weaker to enable any complexation at room temperature. For the former process of complexation involving a charged group, there can be an additional significant driving force from the release of counterions from the associating monomers. Therefore, complexation at lower pH less than 4 and no complexation at higher pH greater than 4 can be expected. Even though the above theoretical argument can be conceptual, and without very detailed accounting of subtleties such as water reorganization and local dielectric constant, it can provide a sound guideline for the observed phenomena outlined herein. It is to be noted that the amine group of MPC could displace the proton of the acrylic acid monomer during complexation, thus making an ion pair in the complexed state. In this situation, the complex can be made more stable due to stronger charge-charge (compared to dipole-charge) interactions and the concurrent gain in translational entropy of the released proton. That being said, the variables can include the pH-tunable charge/dipole moment of the repeat units of the host and guest, ionic strength, and polarizability of the medium. With the above concepts in mind, functional polyzwitterion systems can be designed by selecting for certain properties in these materials.

There can be a need to protect cargo for delivery into the GI tract. Some relevant parameters can include the pH of the stomach, which is close to 2, and the pH of the duodenum, which increases to a value of 5-8. Additionally, the temperature of the GI tract can rest between 35-40° C. With these physiological parameters in mind, various functional chemistries can be anticipated to be useful in this situation.

Using the above guidance from nature, pMPC and pAA can be used to explore the feasibility of this new class of materials, that can include interacting chains of polyzwitterionic and anionic polymers, with respect to their specific applicability to the retention and release of cargo inside the GI tract. Furthermore, pAA can have a simple structure and wide availability, and can also be a common food additive recognized by the United States Food and Drug Administration. In order to survive the harsh conditions inside the stomach, complexation can occur at low pH values, and dissociation can occur at higher pH values.

In addition to this pH-dependent behaviour, complexes can remain intact across a wide temperature range, encompassing ambient conditions in both a lab or household environment, as well as in physiologically-relevant conditions exceeding 35° C. These compounded challenges can serve as an obstacle in orally-delivered drug design, since the exposure of most drugs to the stomach contents can cause rapid disintegration, reducing their uptake efficiency. Hence, blood concentrations of an ingested drug can be lower than the ingested dosage would suggest. Simply raising the dosage is an unacceptable solution for very expensive drugs, or for drugs that cause acute toxicities when present at sufficiently high local concentrations. For these reasons, drug packaging designed expressly for the small intestine (particularly the duodenum) can be a frontier in pharmaceutical research. If cargo did arrive into the small intestine unmolested by the gastric environment, the available blood concentration for that cargo can be much higher than under typical circumstances. Furthermore, if it becomes possible to shield sensitive cargo from corrosion by the stomach, new opportunities can emerge within the context of packaging advanced therapeutics.

### Examples

The systems and methods of the present disclosure include the formulation of a polymer system that undergoes liquid-liquid phase separation to self-assemble into droplets. These polymer systems such as pZC droplets represent a new class of materials distinct from other coacervates, since their composition departs from that of other, oppositely charged polyelectrolytes. Phase behaviour in low-pH conditions that would destroy most electrostatic assemblies, and nonlinear stoichiometry- and pH-dependent behaviour, and spherical morphology in pZC droplets is shown. Next, a mechanism that explains this phenomenology and links these observations to applications in GI physiology can be shown. Finally, a method is described by which the non-equilibrium, responsive properties of these materials was harnessed and viewed in real time using video microscopy, within conditions that mimic relevant aspects of the duodenal environment. pZCs can satisfy the key requirements of GI drug delivery, and provide a blueprint by which strategies for drug delivery into the GI tract can be facilitated.

### 1. Materials and Synthesis

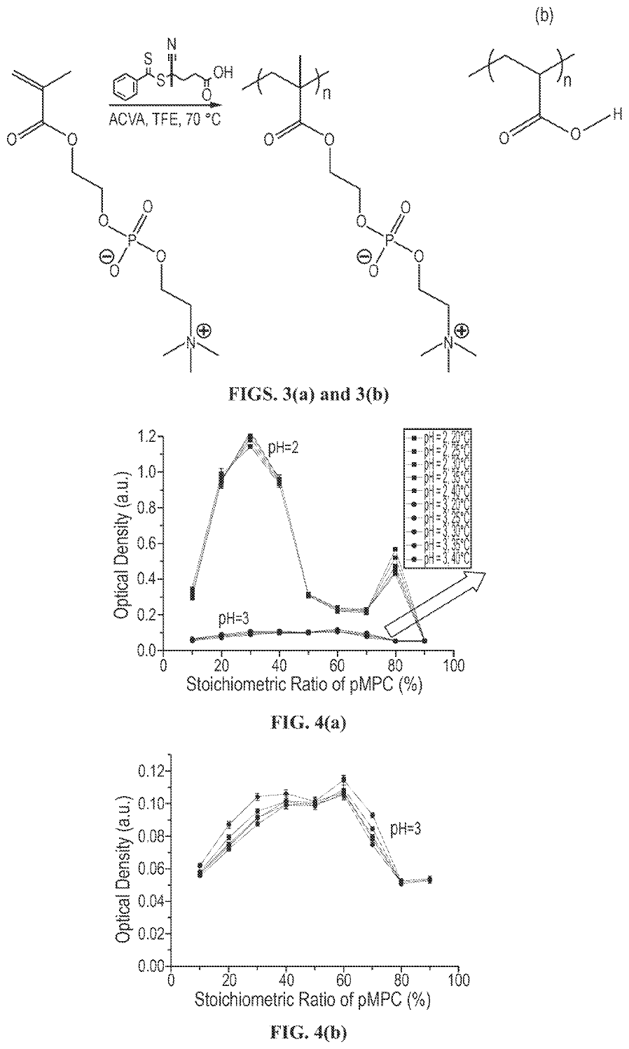

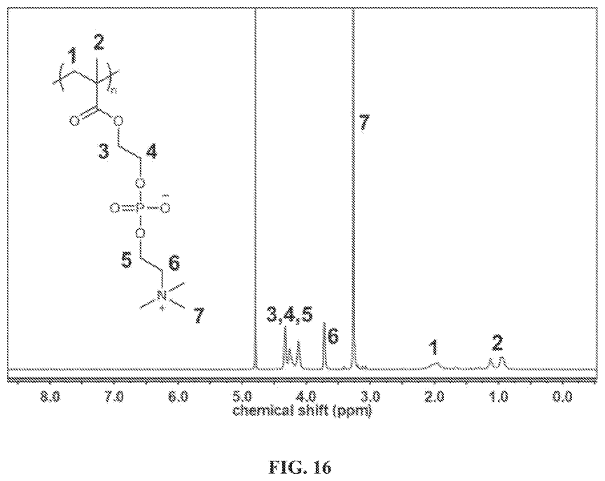

Monomeric MPC was purchased from Sigma Aldrich (MilliporeSigma, Burlington, Massachusetts, USA) (FIG. 3(a)). 1 g of 2-methacryloyloxyethyl phosphorylcholine (MPC) was dissolved in 3 mL of deionized water. To this was added 19 mg 4-cyano-4(phenylcarbonothioylthio)pentanoic acid dissolved in 0.4 mL methanol and 0.1 mL of a 20 mg/mL stock solution of 4,4′-azobis(4-cyanovaleric acid) (ACVA) in methanol. The reaction mixture was degassed by purging with N_{2(g)} under ice bath cooling for 30 minutes. The solution was heated at 70° C. for 6 hours, then quenched by rapidly cooling with liquid nitrogen and opening the reaction vessel to air. The crude product was purified via dialysis against water and subsequently freeze-dried to afford the pure product as a light pink solid in 86% yield with a molecular weight (Mₙ) of 22,000 g/mol. Molecular weight analysis (Mₙ, M_{w} and D) was carried out via gel permeation chromatography (GPC) against PMMA calibration standards, using an Agilent 1200 series system equipped with a degasser, RI-detector, PFG guard column (8×50 mm) and PFG analytical linear M columns (8×300 mm, particle size 7 mm) from Polymer Standards Service, and an isocratic pump. The eluent was 2,2,2-trifluoroethanol (TFE) containing 0.02 M sodium trifluoroacetate, with a flow rate of 1 mL/min at 40° C. ¹H-NMR studies of the synthesized pMPC (500 MHz, D₂O, δ) can reveal the following shifts: 8.05-7.50 (CTA), 4.40-4.00 ppm (6H), 3.71 ppm (2H), 3.27 ppm (9H), 2.20-0.80 (5H+CTA). Spectrum can be found in FIG. 16. pAA (FIG. 3(b)) was purchased (CAS #00627-250, Polysciences, Inc., Warrington, Pennsylvania, USA) as a 25 wt. % solution and diluted in MilliQ water to make a 1 wt. % stock solution. It can have a molecular weight of 50,000 g/mol, and be used without further modification or purification after dilution. HCl (aq) and NaOH (aq) can be used to adjust pH values of stock solutions.

2. Preparation of pAA and pMPC Samples

pAA and pMPC stock solutions were prepared by dissolution of dry polymer powder (pMPC) or concentrated polymer solution (pAA) into Milli-Q water (18.2 MOhm cm resistivity at 25° C., MilliporeSigma, Burlington, Massachusetts, USA). Stock solutions were set to a concentration of 1 weight percent. The stock solution pH was adjusted to a range spanning from 2 to 12 in increments of 1 pH unit using small volumes of concentrated HCl or NaOH. Stock solutions of the pAA and pMPC were mixed at specified stoichiometric ratios in increments of 10% by micropipetting them into microcentrifuge tubes (Thermo Fisher Scientific, Waltham, Massachusetts, USA) containing a specific volume of Milli-Q water to reach a final total polymer concentration of 0.75 wt. %. pMPC was added before the pAA, and this mixing order was kept consistent throughout the measurements. Samples were vortexed at high speed for 10 seconds between each step to ensure thorough and consistent mixing.

### 3. Optical Density

The optical density, or turbidity, or total scattered light intensity of each sample was measured by a Tecan Infinite 200 Pro instrument (Tecan Group Ltd, Männedorf, Switzerland).

Optical density was determined from the equation

$$T = {log}^{1 {0}^{\frac{{I}_{o}}{I}}} ,$$

where Iₒ is intensity of the light unattenuated by the sample, and I is the intensity of the light that is transmitted through the sample. Thus, a reading of T=0.35 can indicate that the sample causes an attenuation of the light intensity by a factor of 10^{0.35}, or 2.24-fold. In other words, only 44.7% (100÷2.24) of the Iₒ light that is incident upon the sample can reach the detector.

Measurements were performed by placing samples within clear, flat-bottomed polystyrene 96-well plates (Corning Inc., Corning, New York, USA), and scanning with 550 nm light, since samples may not absorb light at this wavelength. Each sample was shaken within the plate reader before measurement as a final step to ensure uniform mixing among batches. Turbidity measurements were performed in a temperature range spanning from 20° C. to 40° C. in 5° C. increments.

### 4. Optical Microscopy

Optical micrographs of small aliquots of each sample were recorded on a Leica DM 2700P instrument with a single polarizer (Leica Camera AG, Wetzlar, Germany). Samples were inspected on Fisherbrand glass slides (Thermo Fisher Scientific, Waltham, Massachusetts, USA) under 10× magnification. Phantom PCC 3.5 software (AMETEK Inc. Vision Research, Wayne, New Jersey, USA) was used to compile and visualize still image data, and video micrographs was compiled and processed using the Phantom Video Player software, from the same provider.

### 5. Synthetic Mimic of Dissociation of Complexes on Glass and Video Microscopy

To produce conditions by which a controlled and unidirectional application of a basic medium to a solution of polyzwitterion-polyanion complexes could occur, a setup was constructed to visualize the complex dissociation process. Using a micropipette, a relatively large droplet (300 μL) containing the pZCs was placed at the centre of a glass slide and allowed to relax for several minutes. Next, a much smaller volume of Milli-Q water (15 μL) was added to the edge of the droplet, such that the two fluids could touch and mix. The pipette tip was carefully positioned at the intersection of the droplet with the glass slide, and a sliding motion can be initiated as the plunger was depressed, in order to produce a thin streak of water on the glass. This can ensure that the glass slide is wetted at a specific location, allowing for the subsequent addition of another aqueous solution without surface tension preventing even mixing from occurring.

Next, a similarly small (15 μL) droplet of concentrated NaOH solution was added at the apex of the streak left by the pipette tip when the water was previously deposited onto the glass surface. After addition of the NaOH, it can spontaneously mix with the polyzwitterion-polyanion solution exclusively via this channel in an orderly, unidirectional fashion, since the surface energy mismatch between the glass slide and the concentrated aqueous NaOH was large enough to prevent further wetting of the glass. The progression of the basic medium through the droplet, and the subsequent dissociation of the complexes at the solution front, was monitored using video microscopy by carefully positioning the glass slide underneath the polarized objective such that the velocity vector of the dissociation front intersected the light path of the viewing area.

6. Titration of pAA and pMPC

400 μL of each salt-free polymer solution was prepared by diluting from stock to a concentration of 0.75 wt. % and added to a 2 mL glass vial with a small Teflon stir bar. A pH probe was inserted into the solution, with the tip of the bulb positioned above the bottom of the vial to provide a sufficient amount of space for the stir bar to adequately mix the solution. The titrant was manually added directly into the polymer solution in 1 μl increments using a micropipette, and a sufficient amount of time (1 minute per addition of titrant) can elapse for the solution to equilibrate before the pH value was recorded.

7. Complexation of Protein Cargo within pZC Droplets

Bovine serum albumin (“BSA”) conjugated with a fluorescent dye (Alexa Fluor™) was purchased from Invitrogen (Life Technologies Corporation, Eugene, Oregon, USA) in powdered form. The protein was used as purchased, and dissolved in water. After optimization of loading conditions and concentrations, BSA solution was added in between additions of the pMPC and pAA components of the solution to ensure that complex formation takes place in the presence of protein. Cargo uptake studies were performed by making independent optical density measurements at the peak absorbance wavelength of the protein (495 nm) and that corresponding to the turbidity measurements of the complexes (at 550 nm, as outlined previously in the Methods section) simultaneously. Centrifugation was used to separate the solution into polymer-rich and polymer-poor phases, and the preferential segregation of the cargo was tracked by assessing the absence of protein in the upper polymer-poor phase. (Increased concentrations of protein in the polymer-poor phase can indicate decreased levels of complexation.) Confirmatory microscopy images were taken using a Nikon CrestV2 confocal fluorescence microscope (Nikon Instruments, Inc., Melville, NY, USA).

### A. Liquid-Liquid Phase Separation in Polvzwitterion-Polvelectrolyte Solutions

To assess the phase behavior of poly(2-methacryloyloxyethyl phosphorylcholine)poly(acrylic acid) (structures depicted in FIG. 2, abbreviated as “pMPC-pAA”) droplets, the effect of the mixing ratio of the two components on the turbidity of the resulting solution was tested.

Stock solutions of pMPC and pAA were mixed together over a series of varying stoichiometric ratios. Turbidity measurements, performed at set pH values, was plotted in FIGS. 4(a) and 4(b). At pH=2, a sharp, clearly defined peak (FIG. 4(a)) coincides with a pMPC:pAA stoichiometric ratio of 30:70, indicating that under these conditions, an excess of carboxylic acid can be needed to interact with a given amount of polyzwitterion. This can mean that relatively fewer pAA monomers are available for complexation, as compared to pMPC. Interestingly, a secondary peak at 80:20 can occur at pH 2. At pH 3 (FIG. 4(b), which is a magnification of the results in the bottom trace of FIG. 4(a)), the peak position itself is shifted right, corresponding to a pMPC:pAA stoichiometric ratio of 60:40. This peak is much broader than that in the pH 2 case, which can indicate a reduction in the sensitivity of the pH 3 samples to the mixing stoichiometry.

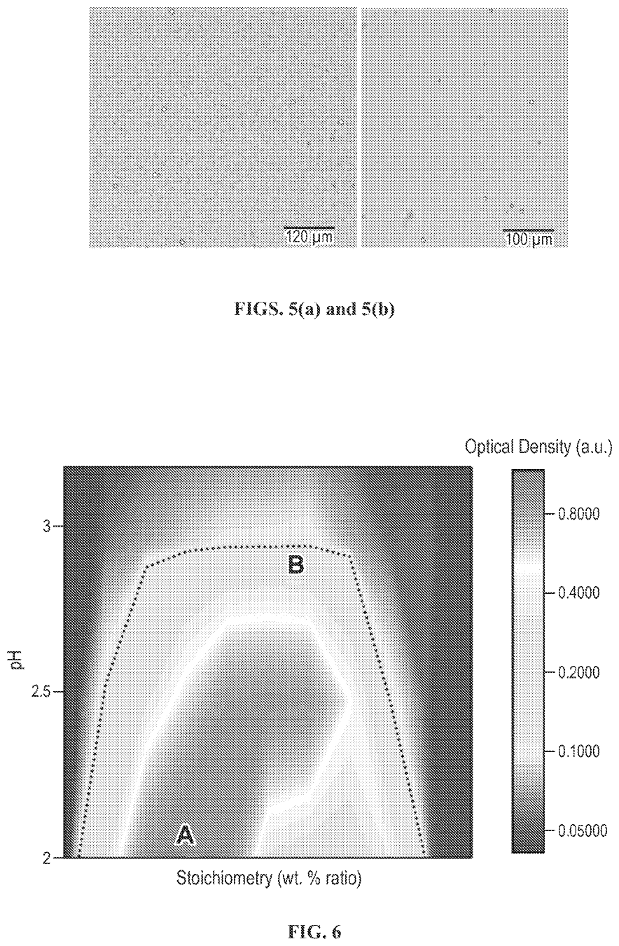

This finding can point to a decrease in the availability of pMPC for complexation at pH 3, since at these conditions, fewer pAA chains may interact with pMPC to produce peak turbidity. Additionally, the relative heights of the turbidity diagrams indicate that more complexation occurs at pH 2 than at pH 3. Quantitatively, it is helpful to note that the relative difference in height between the peak turbidity at pH 2 (30:70 ratio) and at pH 3 (60:40) is approximately one order of magnitude. A cursory comparison of the two micrographs in FIGS. 5(a) and 5(b) shows that the number of droplets in the pH 2 samples far exceeds the number that is seen in the pH 3 samples, which is in agreement with the results from turbidimetry. Furthermore, noting that there is a pH difference of one unit, which can indicate a difference in the proton concentration of an order of magnitude, this result can fit neatly with the idea that the observed phenomenon is a consequence of the ionization equilibria of the constituent chains. For pH values at and exceeding 4, complexation can be completely diminished, and indeed, micrographs at these conditions can indicate the total absence of complexation. These results can be compiled and summarized in the phase diagram depicted in FIG. 6.

A question that may arise from the results thus far is whether the droplets in FIG. 5 contain both pMPC and pAA, or if one component in the solution undergoes self-complexation. To address this point, care can be taken to ensure that both polymers participate in forming droplets. Firstly, a solution of pure pMPC was subjected to increasing concentrations of HCl. As the pH of the solution decreased, there was no change in the turbidity. The turbidity of pAA alone can be insensitive to changes in the pH. pH-induced self-complexation may not take place in either solution component.

Next, the presence of both pMPC and pAA inside the polymer-rich phase can be confirmed, which is corollary to the absence of self-complexation. Upon formulation of a representative solution (e.g., using the peak 30:70 pMPC:pAA mixing ratio), the polymer-rich phase was spun in a centrifuge, and the polymer-poor supernatant was removed. After lyophilizing the solvent from the remaining sample, NMR measurements were done to probe for the presence of each polymer. Evidence from both the pMPC and pAA can be seen in the resulting spectra, which can prove that both chains are participants in the complexation process described herein. These results are summarized in FIG. 15.

Next, to demonstrate that the nature of polyzwitterionic complexation can be sensitive to changes in pH and stoichiometry (as is the case in some polyelectrolyte complex coacervate systems), optical micrographs were systematically recorded over a range of stoichiometric ratios and pH values. There can be significant agreement between the phase diagram plotted in FIG. 6, and the qualitative appearance of the samples under microscopic examination. When present, complexation can be characterized by the presence of mobile, spherical droplets of a highly disperse length scale encompassing the sub-micron to tens-of-microns range.

FIGS. 5(a) and 5(b) can include two representative optical micrographs of polyzwitterionic complexes taken from the samples with the peak turbidity values at pH 2 and 3. The labels A and B in FIG. 6 can correspond to these two samples, namely, the 30:70 sample for pH 2, and the 60:40 ratio for pH 3, respectively. Complexation can be present in almost all samples at pH 2, whereas at pH 3, only some of the samples exhibit spherical droplets, near the peak pH value. These findings bolster the turbidimetry results: the ratios at which complexation is observed can also be those with the highest turbidity values. Furthermore, when comparing pH 3 images to pH 2 images, there can be fewer complexes present in the pH 3 samples, regardless of the stoichiometric ratio. This finding can agree with the correspondingly lower maximum turbidity at pH 3 than at pH 2, since the total scattered light intensity can be responsive both to size and number of scatterers present. Though comparisons of the number of droplets can be made relatively easily between the two images, acquiring robust measurements of droplet size can be nontrivial for numerous reasons. Most importantly, although droplets can form immediately upon mixing, they can also coalesce over time, and the kinetics of this process can be influenced by many factors. Size characterization techniques can be modified significantly to measure quantities such as the R_{g} and Rₕ of chains within these droplets. Measurements of droplets using ImageJ software can yield estimates of the droplet sizes, with the pH 2 sample containing populations of droplets across a very wide distribution of length scales centred around 2.1 microns, and the pH 3 containing far fewer droplets over a narrower length scale centred around 1.65 microns. Lastly, at pH 4, no demonstrable evidence of complexation can be seen at any of the stoichiometric ratios. Hence, there are multiple lines of physical evidence that prove that the phase behaviour of complexes made of pMPC and pAA can be responsive to their chemical milieu, as would be expected from other polyelectrolyte coacervates.

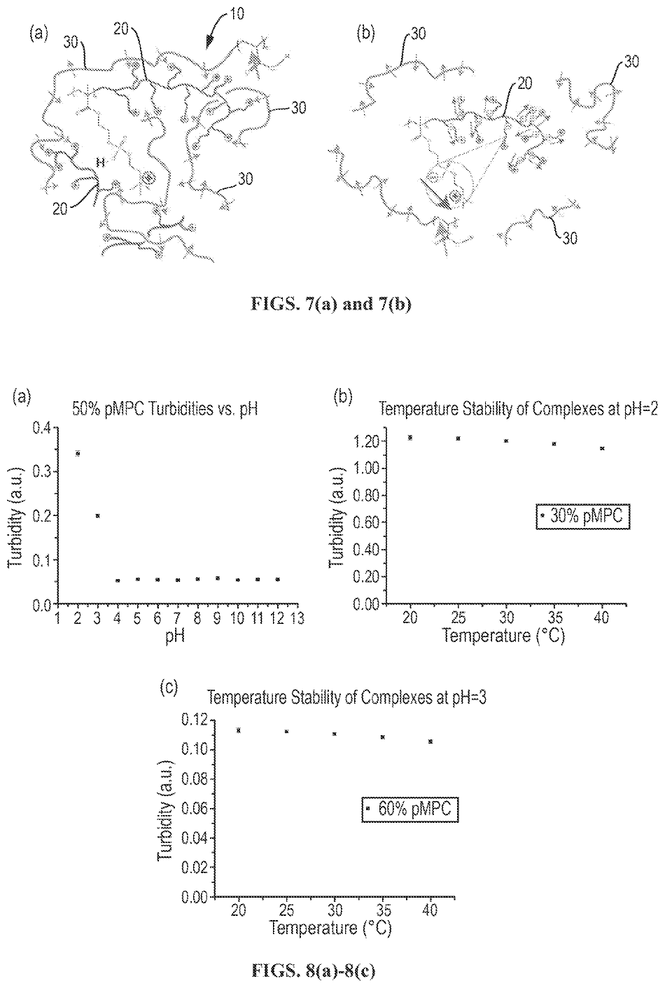

These findings, namely, the non-monotonic dependence of turbidity on the stoichiometric ratio between the two polymers, and the presence of a rich array of spherical droplets of many sizes in numerous samples, can prove that at sufficiently low pH values, pMPC combines with pAA to form a new class of material, analogous to those of the complex coacervate family. Furthermore, their phase behaviour can be orthogonal to that of traditional polyelectrolyte complexes. In other words, at low pH, the pZCs can be stable, but in less acidic conditions, the polymers can no longer interact, leading to disassembly. This behaviour can contrast with that of other polyelectrolyte coacervates, in which complexation only occurs at intermediate pH, and disassembly at very low and very high pH values (FIG. (1)). The mechanism of this orthogonal phase behaviour, depicted in FIGS. 7(a) and 7(b), and its origin in the polyzwitterionic component, can be described in the following section.

### B. Mechanistic Underpinnings of Orthogonal Phase Behaviour of Polyzwitterionic Coacervates

To understand the phase behaviour of pMPC-pAA complexes, the proton association/dissociation equilibrium of each polymer can be considered. This equilibrium dictates the total available charge on each type of polymer chain, which in turn can control the availability of each polymer to undergo complexation with a prospective partner.

In the case of pAA, each chain can either be negatively charged in the deprotonated state, or neutral in the protonated state. The number of chains in either state can be dictated by the pKₐ of the acid group, which can be measured to be 6.2. For pMPC, this equilibrium can be complicated by the fact that there are two charged groups in each monomer. The choline moiety is positively charged, and since it is in a fully quaternized state, it may not undergo typical acid-base reactions. Hence, the choline moiety can carry a permanent positive charge. On the other hand, the phosphoryl group can accept or donate a proton depending on the pH of the solution.

When the pMPC phosphoryl group is deprotonated, its negative charge can neutralize the positively charged choline group. Monomers in this state can contain an effective charge of zero. However, when the phosphoryl group is protonated, its negative charge can be obscured from the choline group. As a result, monomers in this condition can have a formal charge of +1. In solutions that promote the protonation of pMPC, the positively charged choline group can interact with any available pAA chains. The specific pH range in which pMPC is ionized can be dictated by the pKₐ of its phosphoryl group, which can be measured to be 2.3.

Taken together, it is possible to explain the turbidimetry and microscopy results. Using the Henderson-Hasselbalch equation, at a pH of 2, the proportion of pMPC that is positively charged can be close to 80%. The proportion of positively charged monomers of pMPC can decrease as the pH increases, to 28% at pH 2.5, 3.8% at pH 3, and so on. From this trend, it can be apparent why lowering the pH can lead to an increase in complexation. As the pH decreases, more pMPC chains can be available in their positively charged state for interaction with the pAA in the solution.

With respect to the data, the higher scattered light intensity and larger number of visible complexes at pH 2 than at pH 3 can be accounted for by the mechanism described herein. By looking at the proportion of the charged pMPC groups at pH 2 and 3, and factoring in the stoichiometry of the peak samples, there can be a difference of one order of magnitude between the peak turbidities at the two pH values. At pH 2, taking 30% (the peak stoichiometric ratio of pMPC) of the 80% of monomers that are positively charged, 24% of the sample can be composed of charged pMPC units. At pH 3, the peak stoichiometric ratio can be at 60%, and taking that proportion of the 3.8% of the pMPC in a charged state, 2.3% of the overall sample can be composed of charged pMPC monomers. This order-of-magnitude difference (24% vs. 2.3%) can correlate closely with the difference between peak turbidity values of the pH 2 and pH 3 experiments. Furthermore, the rightward shift in the stoichiometry peak, from 30:70 at pH 2 to 60:40 at pH 3, can be accounted for by the decreased charging of the pMPC at higher pH values. Fewer charged groups on pMPC can translate to fewer pAA chains required for interaction. These findings are outlined conceptually in FIGS. 7(a) and 7(b), where FIG. 7(a) shows a polyzwitterionic complex 10 formed of polyelectrolytes 20 and polyzwitterions 30. FIG. 7(b) shows the disruption of the polyzwitterionic complex into its components, namely, a polyelectrolyte 20 and polyzwitterions 30.

The specific nature of the interaction between pMPC and pAA can be found using the energetics of interaction between charges and dipoles. When MPC is at a low pH, it can behave as a cation, and the interaction between it and acrylic acid can be modelled as a charge-dipole interaction. When the ambient pH is raised to 4 and above, the MPC can become dipolar, while the acrylate group can remain dipolar as well. Hence, in this higher pH condition, the energetics can be modelled using dipole-dipole interactions. As shown in FIGS. 2(e) and 2(f), the latter interactions can be almost an order of magnitude weaker. Consequently, stable complexes may not form unless the pH is low enough.

The complexation behaviour of the system can depend most strongly on the ionization of the pMPC component. Although the above explanation is self-consistent, another subtlety may be added to appreciate the data more fully. The process of polyelectrolyte complexation can be entropically driven by the release of counterions into bulk solution. The explanation for the mechanism of complexation in this section hitherto can focus on electrostatic principles that chiefly pertain to enthalpic parameters. These considerations can be important for understanding how the chains look as they associate, and what conditions they may adopt in order to participate in complexation in the first place. To understand why they associate, the above information can be used to describe the entropic contribution to the phase separation process.

Initially, the counter-anion associated with the positively charged amine group on the polyzwitterion can be tethered, preventing these small ions from exploring their full translational entropy in solution. However, as two chains approach one another, the complementary monomer units can interact with one another, instead of their respective counterions, leaving the previously associated counterions free to explore the bulk solution. The complexation of each pair of monomer units can result in an increase in the entropy of the small ions per association. This can yield a net increase in the entropy of the system. This reaction, so to speak, can roughly be summarized as follows:

pMPC-POH—N⁺X⁻+HOOC-pAA→pMPC-POH—N⁺HOOC-pAA+X⁻ (3)

As this reaction proceeds in the forward direction, X⁻ ions can be released into the solution.

This finding can lead us to a consequence of the mechanism described herein. As long as the pMPC can maintain a net positive charge, complexation can occur. However, if pMPC is a chain of pure dipoles with net zero charge, complexation may be disfavoured, and may be a non-starter (N/A). Writing the reaction as before:

pMPC-PO⁻—N⁺+X⁻+HOOC-pAA→N/A (4)

The prospects for phase separation can be absent when pMPC is in a neutral dipolar configuration. There may be nothing to promote forward progress of the reaction above. The pMPC can be uncharged, and thus may not have a negative counterion to release if brought to close proximity to a pAA monomer.

The augmentation of electrostatic attraction by entropy-dependent behaviour of the counterions and chains depicted above can be commonly found in coacervates. The counterion release hypothesis can be the driving force for the complexation between polyelectrolytes. Furthermore, the extension of counterion release as a driving force for polyzwitterionic complex formation can be consistent with existing bodies of evidence, and can also indicate that there may be a certain generalizable universality of this mechanism in driving the phase behaviour of all types of charged macromolecules.

### C. Suitability for Physiological Temperature and pH Criteria

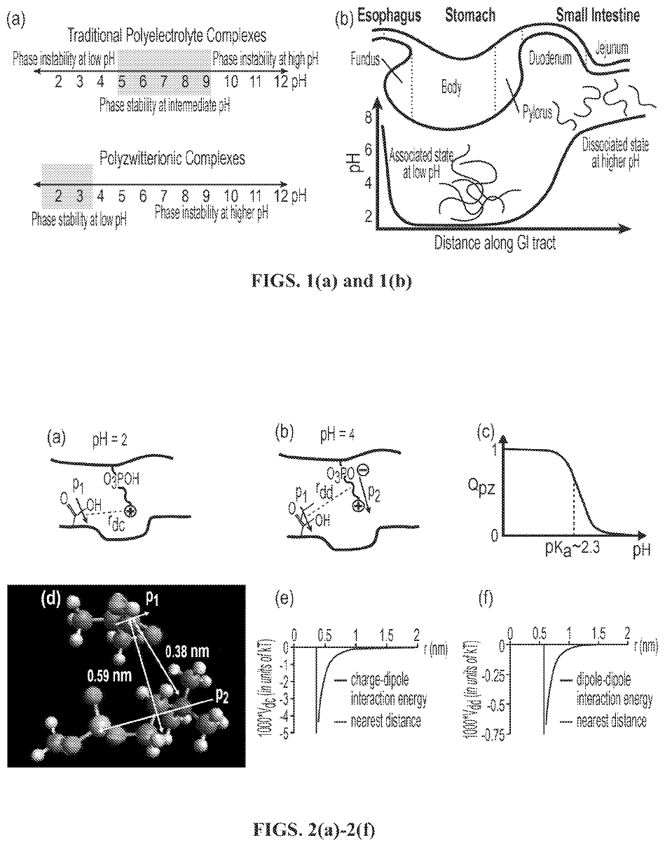

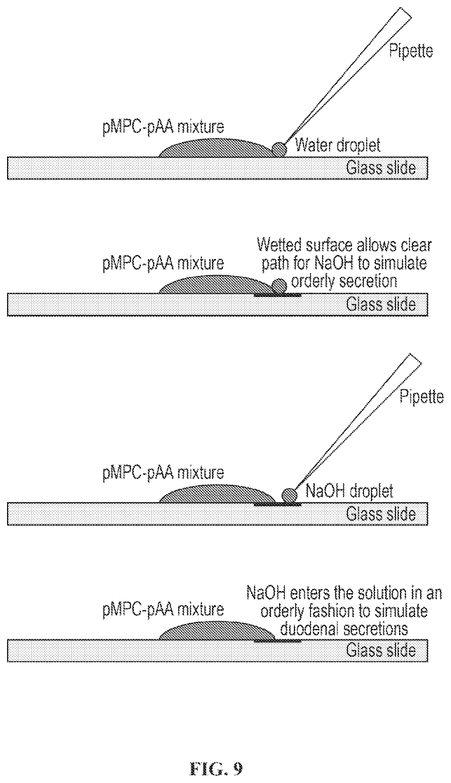

This system can be pertinent in a biomedical context. The system can be tested against three criteria, chosen based on the physiology of the GI tract. Firstly, the system should remain intact at low pH. Second, the system should be unstable at intermediate and high pH values. The latter criterion can account for the environment of the duodenum, in which Brunner's glands can facilitate the secretion of basic fluids that neutralize the acidity of the incoming chyme from the stomach (FIG. 1(b)). Lastly, the complexes should remain intact over a wide temperature range, including that of the GI tract. FIGS. 8(a)-8(c) can demonstrate that the pMPC-pAA complexes can satisfy these three criteria. In FIG. 8(a), high scattered light intensity readings can be observed at low pH, which can indicate stability in the gastric range. A precipitous decline can be seen in the scattered light intensity as the pH reaches 4. Thus, complexation can be poised to occur at the pH of the stomach, followed by dissociation at the higher pH of the duodenal space.

To satisfy the third criterion, pZC temperature stability can be examined by choosing the highest turbidity peak values from the stoichiometry studies in FIG. 4(a), and by varying the temperature of each sample from 20° C. up to 40° C. in 5° C. increments. The results in FIGS. 8(b) and 8(c) can demonstrate that the polyzwitterionic complexes can satisfy the third criterion—the temperature may not exert a strong effect on the levels of complexation between pMPC and pAA. These complexes can largely resist fluctuations in their thermal environment, which can be a condition when considering the possibility that a material used in the GI context may undergo large temperature changes as it travels into and through the body.

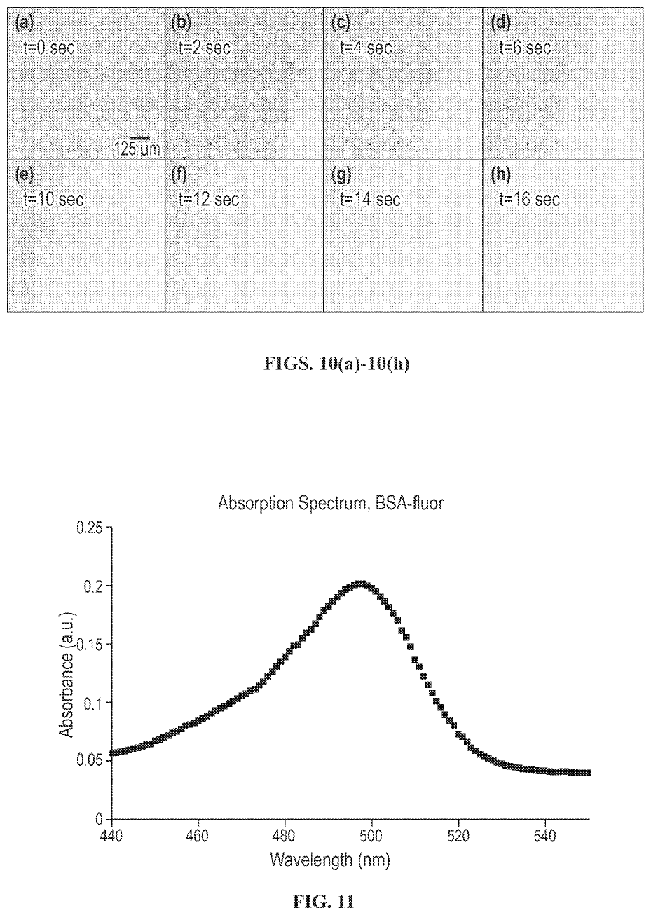

In order to test the non-equilibrium responsiveness of pMPC-pAA complexes to a variable pH, a setup can be constructed (FIG. 9, detailed in the Methods section) that can allow for exposure of the polymers to a basic solution in a controllable fashion. This method was intended to simulate the effects of the secretion of basic buffer by glands in the duodenum. As the basic solution is added to a large droplet containing the pMPC-pAA mixture, it flows from one side of the droplet to the other in a progressive fashion. FIGS. 10(a)-10(h) can summarize videographic evidence of the pH-induced phase instability of the pZC solution. As NaOH travels from right to left, it can dissolve the polymer droplets (sandy, rough-looking area) in its wake. The individual droplets can appear to temporarily expand and rupture, until they are fully decomplexed. Control experiments that replace NaOH solution with pure water do not show any such response of the complexes to the added water, which can prove that this effect is not due simply to dilution, but to the increase in the ambient pH. Interestingly, the pH dependent dissociation can be reversible: after the addition of NaOH to a solution of pZCs, the complexes can dissolve. If HCl is then titrated into the same solution, the pZC droplets can return.

The experiment of FIGS. 10(a)-10(h) can be compared to that of FIG. 6. The two experiments can differ when considering the distinction between equilibrium and non-equilibrium behaviour with respect to pH. Whereas the measurements in FIG. 6 can be made on eleven separate samples prepared over a range of discrete and static pH values, FIGS. 10(a)-10(h) can depict the dissolution of a single sample whose pH was initially set at 2, and then continuously increased by exposure to a basic solution. In other words, FIG. 6 can provide equilibrium information about the pMPC-pAA complexes, while FIGS. 10(a)-10(h) can demonstrate that the spherical polyzwitterionic complexes can exhibit non-equilibrium (with respect to pH) and responsive properties upon exposure to varying physiologically relevant conditions. The activity showcased in FIGS. 10(a)-10(h)—dissolution of the complexes upon exposure to basic media—can highlight the responsive nature of polyzwitterionic complexes, and can point to their potential usefulness as platforms for GI drug delivery.

Having established the above set of activities, cargo can be encapsulated into the pZC droplets. To do this, fluorescently labelled protein, BSA (Bovine Serum Albumin) was used. The protein was added in between the additions of pMPC and pAA, so that droplet formation could occur in the presence of BSA. Solutions prepared in this manner can appear turbid as before, and can carry a slight colour, corresponding to the fluorophore. The ternary solutions were spun in the centrifuge to assess whether the protein co-localizes with the polymer. Optical density measurements of the supernatant (at 495 nm, which is the absorption wavelength of the fluorophore) demonstrate a complete absence of protein in the polymer-poor phase in low pH conditions. By comparison, when the labelled BSA alone was centrifuged in the same spin conditions, absorbance spectra of the conjugated protein was apparent in the solution, which indicates that the protein did not simply sink to the bottom of the tubes during spinning along with the polymers. This experiment was repeated as a function of pH, to assess whether the polyzwitterionic carriers can release their cargo. Absorbance measurements of the supernatant clearly demonstrate the increased upper-phase BSA concentration as the pH increases from 2 to 5. Hence, there is a pH-dependent release of cargo, as can be expected from a carrier that dissolves as a function of pH. Finally, fluorescence micrographs directly demonstrate the localization of the protein into the pZC droplets. These results can be summarized in FIGS. 11-15.

Complexation can exist between pMPC and pAA. These complexes demonstrate many of the same properties as those seen in polyelectrolyte coacervates, even though one of the components is a polyzwitterion, rather than a polyelectrolyte. The presence of the characteristic spherical assemblies seen in coacervate solutions can be confirmed. Their sensitivity to their chemical environment and mixing stoichiometry can be demonstrated. Furthermore, the orthogonal phase behaviour that results from asymmetric charging of the two constituent polymers can be exhibited. A mechanism that explains this behaviour can be described. Lastly, these properties can be harnessed to demonstrate the potential applicability of polyzwitterionic coacervates as platform technologies for the pH-triggered release of cargo.

An implication of the mechanism described herein can be that the specific acid-base chemistry of the monomers, particularly the polyzwitterionic monomer, can dictate the exact range of acid-stability and base-lability of the polymeric complexes. Indeed, different polyzwitterions may yield phase behaviour that is shifted around different pH values relative to that of the pMPC-pAA system at hand. Furthermore, it may be possible to design systems that exhibit inverse phase behaviour relative to what is seen here, namely, base-stability and acid-lability. The feasibility of engineering such phenomena using complementary synthetic approaches can be envisioned.

Aspects of the phase behaviour presented herein can have implications in complexation within other polyzwitterionic materials. Designing analogous synthetic polyzwitterions to tailor specific pH-responsive criteria could be one avenue of fruitful research. The chemistry can be adjusted to suit stability or instability requirements at particular pH values. Furthermore, switching the two charged groups on the polyzwitterionic monomer, such that the permanently charged functional group is negative, and the variably charged functional group is positive, can lead to inverse phase behaviour relative to that described in this work, if such a polyzwitterion were paired with a polycation. The findings can pertain also to naturally-occurring polyzwitterionic materials, namely proteins. Designing conjugates or adjuvants that interact with proteins to promote self-assembling structures can take into account the phenomena and concepts described and outlined herein.

In more applied contexts, the type of pH-dependent phenomenology described herein can be engineered into existing modalities of drug transport and delivery. Furthermore, in the relatively chaotic environment of the GI tract, the influence of additional factors, such as other complexing agents, and physiological parameters beyond the pH and temperature (such as ionic strength), should be taken into account. The systems and methods of the present disclosure can lay the groundwork to tackle the imminent problem in pharmacology of GI drug delivery, among other problems for which the present teachings and pZCs may solve.

### D. Encapsulation of Model Cargo

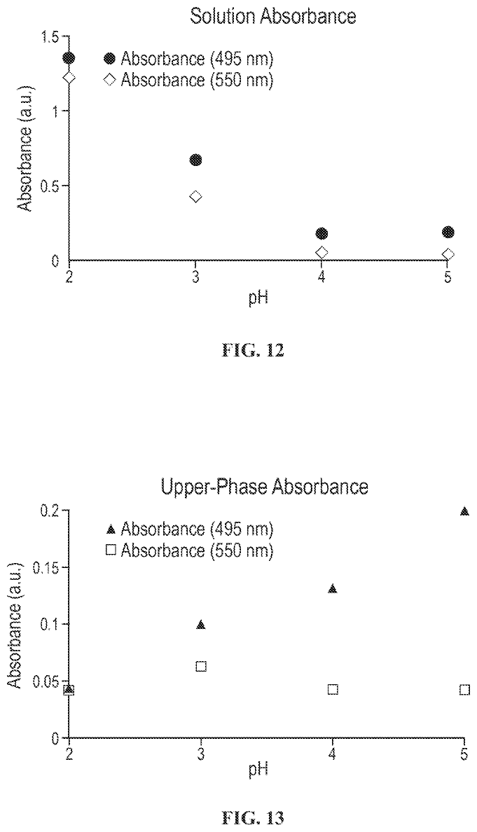

These experiments demonstrate the encapsulation of model cargo, BSA, into the pZC droplets. First, the absorbance spectrum of fluorescently labeled BSA was measured to determine its peak absorption wavelength (495 nm). The fluorophore was selected such that it has an absorption peak sufficiently distant from the wavelength at which turbidity measurements were collected (550 nm). Next, the three-component system was mixed to yield a solution containing both the protein as well as the pZC droplets. Encouragingly, the bulk solution absorbance in the presence of BSA follows the same pH-dependent behavior seen in the absence of BSA. However, this measurement may not sufficiently localize the BSA to the interior or exterior (or both) of the pZC droplets. In order to test if/where the protein segregates within the solution, the samples were spun down in the centrifuge to induce bulk phase separation, into a polymer-rich lower phase and a polymer-poor supernatant. Because accurate measurement of the turbidity of a viscous and low-volume lower phase can be experimentally difficult, absorbance measurements can be carried out on the upper phase instead. At pH 2, when complexes form, the upper phase turbidity at 550 nm (corresponding to the polymer droplets) and the 495 nm absorbance can be at their minimum baseline value, indicating the absence of both the protein and the complexes. This finding can indicate that the protein co-localizes with the polyzwitterion complexes at low pH. However, as the pH was increased to 4 and 5, the upper phase absorption at 495 nm can reach levels that correspond to the bulk solution absorbance of the protein of −0.2 a.u., which is consistent with the value of the peak seen in FIG. 11. This finding can indicate that as the pH is increased, the protein is released, and thus cannot segregate with the polyzwitterionic complexes. Bulk solutions of BSA-loaded pZCs can have the highest absorbance at low pH as shown in FIG. 12. FIG. 13 shows the upper-phase absorbance after centrifugation as a function of pH.

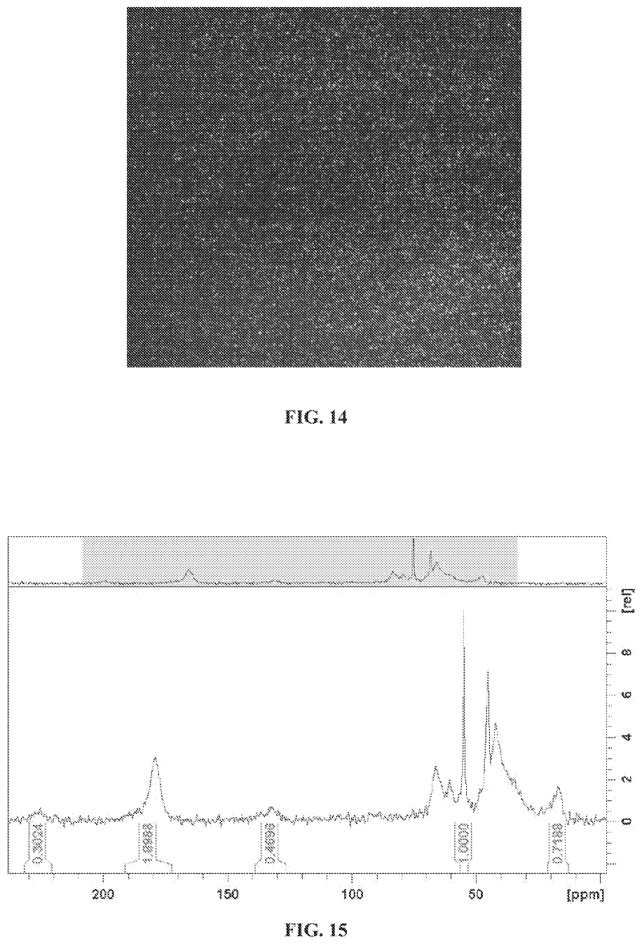

Finally, to test the preferential segregation of BSA with the polyzwitterionic complexes directly, fluorescence microscopy can be performed to observe the co-localization of the proteins and the chains into droplets. A representative image can be seen in FIG. 14.

### E. NMR Study

Upon formulation of a representative solution (the peak 30:70 pMPC:pAA mixing ratio at pH 2), the polymer-rich phase can be spun in a centrifuge, and the polymer-poor supernatant can be removed. After lyophilizing the solvent from the remaining sample, solid state NMR measurements probed for the presence of each polymer. As expected, evidence from both pMPC and pAA is seen in the resulting spectra, which shows that both chains are participants in the complexation process. 10% of D₂O added to the solid to enhance the spectral resolution. Cross-polarization (CP) NMR was used to obtain the spectrum in FIG. 15 and ¹H-NMR was used to obtain the spectrum in FIG. 16.

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

We claim:

1. A polymer system, wherein the polymer system comprises a polyelectrolyte and a polyzwitterion whereby the polyelectrolyte and the polyzwitterion self-assemble to form a polyzwitterionic complex.

2. The polymer system of claim 1, wherein the polyzwitterionic complex encapsulates a cargo.

3. The polymer system of claim 2, wherein the cargo is a therapeutic drug product.

4. The polymer systems of claim 3, wherein the therapeutic drug product is selected from the group consisting of a diabetes medication, insulin, a glucagon-like peptide-1 receptor agonist, a gastric inhibitory peptide, a probiotic agent, a non-steroidal anti-inflammatory drug, a vaccine, a monoclonal antibody, and a nucleic acid.

5. The polymer system of claim 1, wherein the polyelectrolyte is a pharmaceutically acceptable polyelectrolyte and the polyzwitterion is a pharmaceutically acceptable polyzwitterion.

6. The polymer system of claim 1, wherein the polyzwitterion is grafted to a substrate.

7. The polymer system of claim 1, wherein at least one of the polyelectrolyte and the polyzwitterion is a co-polymer.

8. The polymer system of claim 1, wherein the polyelectrolyte is selected from the group consisting of poly(acrylic acid), poly(styrene sulfonic acid), poly(methyl methacrylate), poly(dimethyl amino ethyl methacrylate), poly(glutamic acid), poly(aspartic acid), heparan sulfate, hyaluronic acid, and co-polymers thereof.

9. The polymer system of claim 1, wherein the polyzwitterion is selected from the group of polyzwitterion classes consisting of a phosphorylcholine class, a sulfobetaine class, a carboxybetaine class, an ammonium sulfonate class, a sulfopyridinium betaine class, a cysteine class, and co-polymers thereof.

10. The polymer system of claim 1, wherein the polyzwitterion is poly(2-methacryloyloxyethyl phosphorylcholine).

11. The polymer system of claim 1, wherein the polymer system further comprises a solvent, optionally, wherein the solvent comprises water, methanol, ethanol, ethylene glycol, propylene glycol, polyethylene glycol, or combinations thereof.

12. (canceled)

13. The polymer system of claim 11, wherein the solvent comprises one or more of sodium chloride, potassium chloride, a sugar, hydrochloric acid, sulfuric acid, nitric acid, hydrobromic acid, and acetic acid.

14. The polymer system of claim 1, wherein the polymer system is in a solid state.

15. A reconstituted polymer system comprising the polymer system of claim 14; and water.

16. The polymer system of claim 1, wherein the polyzwitterionic complex is formed at a pH of about 1 to about 3.

17. A polymer system comprising a coacervate or precipitate formed through dipole-charge interactions or dipole-dipole interactions.

18. The polymer system of claim 17, comprising a polyelectrolyte and a polyzwitterion.

19. A method of using a polymer system to deliver a cargo, wherein the polymer system comprises a polyzwitterionic complex self-assembled from a polyelectrolyte and a polyzwitterion and encapsulates a cargo whereby when the pH of the surrounding environment changes, the polyzwitterionic complex is undone thereby releasing the cargo.

20. A method of claim 19, wherein the pH of the surrounding environment is increased to release the cargo.

21. The polymer system of claim 19, wherein the cargo is a therapeutic drug product.
